(19) United States
(12) Patent Application Publication
(10) Pub. No.: US 2024/0252680 A1
(43) Pub. Date: Aug. 1, 2024

(54) COMPOSITION FOR INHIBITING ALPHA-SYNUCLEIN AGGREGATION AND METHOD FOR INHIBITING ALPHA-SYNUCLEIN AGGREGATION

(71) Applicant: INNOPEUTICS CORPORATION, Seoul (KR)

(72) Inventors: Sang Hun LEE, Seoul (KR); Yun Seon YANG, Seoul (KR); Min Jong SEOK, Seoul (KR); Tae Gyun KIM, Seoul (KR); Seonghoon LEE, Seoul (KR)

(21) Appl. No.: 18/290,124

(22) PCT Filed: Apr. 21, 2022
(86) PCT No.: PCT/KR2022/005687

§ 371 Date: Nov. 9, 2023

(30) Foreign Application Priority Data
Jul. 14, 2021 (KR) 10-2021-0092446
Nov. 24, 2021 (KR) 10-2021-0163358

(51) Int. Cl. A61K 48/00; A61K 35/30; A61K 38/17; A61P 25/16; C12N 15/86

(52) U.S. Cl. CPC A61K 48/005; A61K 35/30; A61K 38/1709; A61P 25/16; C12N 15/86; C12N 2750/14143

(57) ABSTRACT

The present invention relates to a composition for inhibiting alpha-synuclein aggregation and a method for inhibiting alpha-synuclein aggregation and, more specifically, to techniques for inhibiting alpha-synuclein aggregation and phosphorylation by introducing Nurr1 and Foxa2 genes to induce the expressions thereof, and the composition according to the present disclosure has an excellent effect of inhibiting α-synuclein aggregation and phosphorylation and thus can be used in the treatment and prevention of Parkinson's disease.

### TECHNICAL FIELD

The present disclosure relates to a composition for inhibiting alpha-synuclein aggregation and a method for inhibiting alpha-synuclein aggregation and, more specifically, to techniques for inhibiting alpha-synuclein aggregation and phosphorylation by introducing Nurr1 and Foxa2 genes to induce the expressions thereof.

### BACKGROUND ART

Parkinson's disease is a neurodegenerative disorder associated with motor disturbances, such as the muscle tremors and muscular stiffness that occur at the onset of the disease. Parkinson's disease mainly occurs in the elderly, and it is known that the risk of Parkinson's disease increases as the age of a subject increases. In South Korea, approximately 1 to 2 people per 1000 people are estimated to have Parkinson's disease, and most cases of Parkinson's disease occurring in the elderly are not known to be strongly influenced by genetic factors. Parkinson's disease is known to be caused by the death of dopaminergic cells in an area called the substantia nigra in the midbrain, but exact reasons for the destruction of dopaminergic cells in the substantia nigra are not currently known. It has recently been predicted that as the average life expectancy of humans increases, the frequency of Parkinson's disease will also increase.

The management and treatment of Parkinson's disease incurs a huge cost, and the mental suffering of patients is also considerable. Therefore, effective methods for prevention and treatment of Parkinson's disease are needed.

Recently, many studies are focused on the alpha-synuclein (α-synuclein) protein associated with Parkinson's disease. α-synuclein is an abundant protein in the human brain and is mainly found in specially structured ends of neurons, called presynaptic terminals. Previous studies have established that Parkinson's disease is associated with the formation of aggregates of α-synuclein and the formation of Lewy bodies due to a disruption in the balance between the generation and removal of α-synuclein inside neurons. Lewy bodies are known to cause an influx of calcium ions due to modification of neuronal permeability. Additionally, Lewy bodies are known to induce oxidative stress due to mitochondrial damage and to also interfere with normal microtubule formation. These pathological processes result in neuronal death, thereby contributing to the development of Parkinson's disease. In this context, attempts to inhibit α-synuclein are being conducted. However, a therapeutic agent or method capable of effectively inhibiting the aggregation of α-synuclein has not yet been developed.

### DISCLOSURE OF INVENTION

### Technical Problem

Hence, the present inventors reveal herein that the aggregation of α-synuclein protein is inhibited when genes for the transcription factors Nurr1 and Foxa2 are introduced and expressed in brain cells. It was discovered that the combinative expression of Nurr1 with the co-activator Foxa2 rather than the expression of Nurr1 alone had strong α-synuclein protein aggregation inhibitory effects through synergistic effects.

Accordingly, an aspect of the present disclosure is to provide a composition for inhibiting α-synuclein protein aggregation, the composition containing a gene carrier containing Nurr1 and Foxa2 genes.

Accordingly, an aspect of the present disclosure is to provide an α-synuclein protein aggregation inhibitor containing a gene carrier containing Nurr1 and Foxa2 genes.

Another aspect of the present disclosure is to provide a composition for inhibiting α-synuclein protein aggregation, the composition containing a vector loaded with Nurr1 and Foxa2 genes.

Still another aspect of the present disclosure is to provide a composition for inhibiting α-synuclein protein aggregation, the composition containing a vector loaded with Nurr1 and Foxa2 genes.

Still another aspect of the present disclosure is to provide a composition for inhibiting α-synuclein protein aggregation, the composition containing brain cells transduced with Nurr1 and Foxa2 genes.

Still another aspect of the present disclosure is to provide an α-synuclein protein aggregation inhibitor containing brain cells transduced with Nurr1 and Foxa2 genes.

Still another aspect of the present disclosure is to provide a composition for inhibiting α-synuclein protein phosphorylation, the composition containing a gene carrier containing Nurr1 and Foxa2 genes.

Still another aspect of the present disclosure is to provide an α-synuclein protein phosphorylation inhibitor containing a gene carrier containing Nurr1 and Foxa2 genes.

Still another aspect of the present disclosure is to provide a composition for inhibiting α-synuclein protein phosphorylation, the composition containing a vector loaded with Nurr1 and Foxa2 genes.

Still another aspect of the present disclosure is to provide an α-synuclein protein phosphorylation inhibitor containing a vector loaded with Nurr1 and Foxa2 genes.

Still another aspect of the present disclosure is to provide a composition for inhibiting α-synuclein protein phosphorylation, the composition containing brain cells transduced with Nurr1 and Foxa2 genes.

Still another aspect of the present disclosure is to provide an α-synuclein protein phosphorylation inhibitor containing brain cells transduced with Nurr1 and Foxa2 genes.

Still another aspect of the present disclosure is to provide a composition for the prevention or treatment of a disease caused by α-synuclein protein aggregation, the composition containing a gene carrier containing Nurr1 and Foxa2 genes.

Still another aspect of the present disclosure is to provide a composition for the prevention or treatment of a disease caused by α-synuclein protein aggregation, the composition containing a vector loaded with Nurr1 and Foxa2 genes.

Still another aspect of the present disclosure is to provide a composition for the prevention or treatment of a disease caused by α-synuclein protein aggregation, the composition containing brain cells transduced with Nurr1 and Foxa2 genes.

### Solution to Problem

The present inventors conducted intensive research into methods for inhibiting α-synuclein protein aggregation, which is known to be a main cause of Parkinson's disease. As a result, it was established that the transduction and expression of Nurr1 and Foxa2 genes, compared with the transduction and expression of Nurr1 gene alone, can better inhibit the aggregation and phosphorylation of α-synuclein protein.

In accordance with an aspect of the present disclosure, there is provided a composition for inhibiting α-synuclein protein aggregation, the composition containing a gene carrier containing Nurr1 and Foxa2 genes.

In accordance with an aspect of the present disclosure, there is provided an α-synuclein protein aggregation inhibitor containing a gene carrier containing Nurr1 and Foxa2 genes.

In accordance with another aspect of the present disclosure, there is provided a composition for inhibiting α-synuclein protein aggregation, the composition containing a vector loading Nurr1 and Foxa2 genes.

In accordance with still another aspect of the present disclosure, there is provided an α-synuclein protein aggregation inhibitor containing a vector loading Nurr1 and Foxa2 genes.

In accordance with still another aspect of the present disclosure, there is provided a composition for inhibiting α-synuclein protein aggregation, the composition containing brain cells transduced with Nurr1 and Foxa2 genes.

In accordance with still another aspect of the present disclosure, there is provided an α-synuclein protein aggregation inhibitor containing brain cells transduced with Nurr1 and Foxa2 genes.

In accordance with still another aspect of the present disclosure, there is provided a composition for inhibiting α-synuclein protein aggregation, the composition containing any one selected from the group consisting of: a gene carrier containing Nurr1 and Foxa2 genes; and brain cells transduced with Nurr1 and Foxa2 genes.

In accordance with still another aspect of the present disclosure, there is provided a composition for inhibiting α-synuclein protein aggregation, the composition containing any one selected from the group consisting of: a vector loading Nurr1 and Foxa2 genes; and brain cells transduced with Nurr1 and Foxa2 genes.

In accordance with still another aspect of the present disclosure, there is provided an α-synuclein protein aggregation inhibitor containing any one selected from the group consisting of: a gene carrier containing Nurr1 and Foxa2 genes; and brain cells transduced with Nurr1 and Foxa2 genes.

In accordance with still another aspect of the present disclosure, there is provided an α-synuclein protein aggregation inhibitor containing any one selected from the group consisting of: a viral vector loading Nurr1 and Foxa2 genes; and brain cells transduced with Nurr1 and Foxa2 genes.

In accordance with still another aspect of the present disclosure, there is provided a composition for inhibiting α-synuclein protein phosphorylation, the composition containing a gene carrier containing Nurr1 and Foxa2 genes.

In accordance with still another of the present disclosure, there is provided an α-synuclein protein phosphorylation inhibitor containing a gene carrier containing Nurr1 and Foxa2 genes.

In accordance with still another aspect of the present disclosure, there is provided a composition for inhibiting α-synuclein protein phosphorylation, the composition containing a vector loading Nurr1 and Foxa2 genes.

In accordance with still another aspect of the present disclosure, there is provided an α-synuclein protein phosphorylation inhibitor containing a vector loading Nurr1 and Foxa2 genes.

In accordance with still another aspect of the present disclosure, there is provided a composition for inhibiting α-synuclein protein phosphorylation, the composition containing brain cells transduced with Nurr1 and Foxa2 genes.

In accordance with still another aspect of the present disclosure, there is provided an α-synuclein protein phosphorylation inhibitor containing brain cells transduced with Nurr1 and Foxa2 genes.

In accordance with still another aspect of the present disclosure, there is provided a composition for inhibiting α-synuclein protein phosphorylation, the composition containing any one selected from the group consisting of: a gene carrier containing Nurr1 and Foxa2 genes; and brain cells transduced with Nurr1 and Foxa2 genes.

In accordance with still another aspect of the present disclosure, there is provided a composition for inhibiting α-synuclein protein phosphorylation, the composition containing any one selected from the group consisting of: a vector loading Nurr1 and Foxa2 genes; and brain cells transduced with Nurr1 and Foxa2 genes.

In accordance with still another aspect of the present disclosure, there is provided an α-synuclein protein phosphorylation inhibitor containing any one selected from the group consisting of: a gene carrier containing Nurr1 and Foxa2 genes; and brain cells transduced with Nurr1 and Foxa2 genes.

In accordance with still another aspect of the present disclosure, there is provided an α-synuclein protein phosphorylation inhibitor containing any one selected from the group consisting of: a viral vector loading Nurr1 and Foxa2 genes; and brain cells transduced with Nurr1 and Foxa2 genes.

As used herein, the term “α-synuclein protein” is one of the proteins abundant in the brain and is mainly found in the ends of nerve cells called presynaptic terminals. The α-synuclein is known to interact with phospholipids and proteins and to help regulate the release of the neurotransmitter dopamine. Human α-synuclein consists of about 140 amino acids and is encoded by the SNCA gene.

As used herein, “α-synuclein protein aggregation” refers to the aggregation of two or more α-synuclein proteins. In an embodiment of the present disclosure, the α-synuclein aggregate may have a larger molecular weight and/or size compared with the non-aggregated α-synuclein protein.

As used herein, the term “inhibiting α-synuclein protein aggregation” may be understood to mean inhibiting the formation of an aggregate formed through the aggregation of α-synuclein protein with neighboring α-synuclein proteins, or breaking down aggregate that has already formed. Alternatively, the inhibiting of α-synuclein protein aggregation does not mean only inhibiting aggregation, but also may encompass increasing the rate of breakdown of α-synuclein protein or its aggregates, or controlling the balance between the formation and breakdown rates of α-synuclein or its aggregates to a normal state.

As used herein, the term “gene carrier” refers to a means for delivering a nucleic acid sequence or a composition containing a nucleic acid sequence to a cell or tissue. For example, examples of the gene carrier may include viral vectors or non-viral vectors (e.g., carries based on retroviruses, adenoviruses, adeno-associated viruses, and other nucleic acids), injection or microinjection of naked nucleic acids, polymer-based delivery systems (e.g., liposomes and metallic particle systems), biolistic injection lipid nanoparticles (LNP), and the like, but are not limited thereto.

In an embodiment of the present disclosure, the gene carrier may be a viral vector.

As used herein, the term “brain cells” refers to cells located in the brain, and examples of the brain cells may include neurons (neuronal cells) and glia (glial cells).

As used herein, the term “neurons” refers to cells of the nervous system. As used herein, the term “neurons” may be used exchangeably with “nerve cells” and “neuronal cell”.

As used herein, the term “glia” refers to cells that occupy the largest part of cells present in the brain, and the glia may include astrocytes or microglia. The astrocytes are involved in neuron protection, nutrition supply, and inflammation, and the microglia are responsible for inflammation in the brain, and these cells are known to play an important role in brain diseases, such as Alzheimer's disease.

As used herein, the term “transduction” refers to an introduction of a genetic trait resulting from the transferring of the genetic trait from one cell to another cell via a bacteriophage, and in some cases, the infection of a certain type of bacteria with bacteriophages results in the binding of phage DNA to host DNA, and the phages holding some of the host DNA instead of losing some of their own DNA are released through cell lysis. The infection of other bacteria with these phages results in the new introduction of a gene of the previous host, thereby showing a new trait. The term “transduction” in biologic research commonly indicates the introduction and expression of a specific exogenous gene in target cells using viral vector(s).

As used herein, the term “cell therapeutic agent” refers to a drug used for the purpose of treatment, diagnosis, and prevention, which contains a cell or tissue prepared through isolation from man, culture and specific operation (as provided by the USFDA), and specifically, the term refers to a drug used for the purpose of treatment, diagnosis, and prevention through a series of behaviors of in vitro multiplying and sorting living autologous, allogenic and xenogenic cells or changing the biological characteristics of cells in order to recover the functions of cells and tissues. Cell therapeutic agents are broadly divided, according to the differentiation level of cells, into somatic cell therapeutic agents and stem cell therapeutic agents.

As used herein, the term “introduce (transduce) Nurr1 and Foxa2” refers to introducing nucleic acids encoding the two genes together into brain cells. The two genes may be introduced through gene carrier(s) separately or simultaneously. When a vector is used as a gene carrier, the two genes may be introduced by respective expression vectors, separately, or by a single expression vector, simultaneously.

It was identified in an example of the present disclosure that the introduction of both Nurr1 and Foxa2 genes showed significant α-synuclein protein aggregation inhibitory ability due to synergistic effects between Nurr1 and Foxa2, compared with the introduction of Nurr1 or Foxa2 gene alone. Specifically, it was identified that the introduction of both Nurr1 and Foxa2 genes into cells significantly reduced both α-synuclein protein monomers and aggregates compared with the introduction of Nurr1 or Foxa2 gene alone (see FIGS. 5 and 6).

As used herein, the term “introduce (transduce) Nurr1” refers to introducing a nucleic acid encoding Nurr1 gene into brain cells.

In order to introduce genes encoding Nurr1 and/or Foxa2 into brain cells, an intracellular introduction technique through a gene carrier known in the art may be used, for example, a viral vector may be used, such as using adeno-associated virus (AAV), retrovirus, or adenovirus.

Viral vectors may be loaded with Nurr and/or Foxa2. The viral vectors may employ adeno-associated virus (AAV), adenovirus, retrovirus, and/or lentivirus, but are not limited thereto. Therefore, in an embodiment, the introduction of Nurr1 and/or Foxa2 according to the present disclosure may include inserting nucleic acids encoding Nurr1 and/or Foxa2 into separate individual expression vectors or one expression vector and then introducing the expression vector or vectors into brain cells.

The respective nucleic acids encoding Nurr1 and/or Foxa2 may be used without limitation as long as the nucleic acids have nucleotide sequences encoding Nurr1 and/or Foxa2, known in the art. Also, the nucleic acids may have nucleotide sequences encoding respective functional equivalents of Nurr1 and/or Foxa2. The functional equivalent refers to a polypeptide having a sequence homology (that is, identity) of at least 60%, preferably at least 70%, more preferably at least 80% to the amino acid sequence of Nurr1 and/or Foxa2. For example, the functional equivalent includes a polypeptide having a sequence homology of 60%, 61%, 62%, 63%, 64%, 65%, 66%, 67%, 68%, 69%, 70%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and 100%. The functional equivalent may be generated as a result of addition, substitution, or deletion of a part of the amino acid sequence. The deletion or substitution of amino acids may occur at sites that are not directly associated with physiological activities of the polypeptide of the present disclosure.

In addition, the nucleic acids encoding Nurr1 and/or Foxa2 may be prepared using genetic recombination methods known in the art. For example, the nucleic acids encoding Nurr1 and/or Foxa2 may be prepared using PCR amplification for amplifying nucleic acids from genomes, chemical synthesis, or cDNA synthesis.

The nucleic acids encoding Nurr1 and/or Foxa2 may be operably linked to an expression control sequence and thus can be inserted into expression vectors. The term “operably linked” means that one nucleic acid fragment is linked to another nucleic acid fragment and thus the functions or expressions of the one nucleic acid fragment are affected by the other nucleic acid fragment. In addition, the term expression control sequence refers to a DNA sequence that controls the expression of an operably linked nucleic acid sequence in a particular host cell. Such a control sequence may include a promoter for initiating transcription, any operator sequence for controlling transcription, a sequence for encoding a suitable mRNA ribosomal binding site, and a sequence for controlling the termination of transcription and translation. All of these sequences may be generally expressed as a “DNA construct containing nucleic acids encoding Nurr1 and/or Foxa2.

As used herein, the term “expression vector” refers to a viral vector or other vehicle known in the art, into which a nucleic acid encoding a structural gene may be inserted and which may enable the nucleic acid to be expressed in a host cell.

In the present disclosure, the vector may be a viral vector. The viral vector may be an adeno-associated viral (AAV) vector, a retroviral vector, an adenoviral vector, a lentiviral vector, a herpes virus vector, an avipoxvirus vector, or the like, but is not limited thereto.

The adeno-associated viral (AAV) vector may be constructed by transducing materials capable of making virus into a specific cell. The lentiviral vector may also be constructed through several steps to produce virus in a specific cell line.

The expression vector containing a nucleic acid according to the present disclosure may be introduced into brain cells by a known method for introducing the nucleic acid into the cells by means of a method known in the art, for example, viral transduction, transient transfection, or microinjection, but is not limited thereto. For example, Nurr1 and/or Foxa2 are inserted into the adeno-associated viral (AAV) or lentiviral vector by gene recombination technology to construct an expression vector, and then this vector is transduced in a packaging cell, and the transduced packaging cell is cultured, followed by separation and purification, thereby obtaining an AAV or lentiviral solution. Then, the solution may be used to infect brain cells (neurons and/or glia) to introduce the Nurr1 and/or Foxa2 genes into the brain cells. Subsequently, the expression of Nurr1 and/or Foxa2 alone or in combination is investigated by using a selective marker contained in the AAV or lentiviral vector, and then desired brain cells can be obtained.

The brain cells having Nurr1 and Foxa2 transduced and expressed therein according to the present disclosure may be prepared by a method including the following steps:
a preparation step of preparing a recombinant gene carrier containing a DNA construct containing nucleic acids encoding Nurr1 and Foxa2; and
a transfection step of transfecting brain cells with the gene carrier containing Nurr1 and Foxa2.

The brain cells having Nurr1 and Foxa2 transduced and expressed therein according to the present disclosure may be prepared by a method including the following steps:
a preparation step of preparing a recombinant viral vector containing a DNA construct containing nucleic acids encoding Nurr1 and Foxa2;
a production step of transfecting a virus-producing cell line with the recombinant viral vector to prepare a Nurr1- and Foxa2-expressing recombinant virus; and
a transduction step of infecting brain cells with the Nurr1- and Foxa2-expressing recombinant virus.

A DNA construct is operably linked to an expression control sequence, for example, a promoter, and inserted into a viral vector known in the art, thereby constructing a recombinant viral vector. Thereafter, the recombinant viral vector containing the nucleic acids encoding Nurr1 and/or Foxa2 is introduced into a virus-producing cell line, thereby preparing a recombinant virus expressing Nurr1 and Foxa2. A cell line producing a virus corresponding to the desired viral vector may be used as the virus-producing cell line. Then, brain cells are infected with the recombinant AAV or lentivirus expressing Nurr1 and Foxa2 or Nurr1. This may be carried out by using a method known in the art.

The brain cells expressing Nurr1 and/or Foxa2 according to the present disclosure may be multiplied and cultured by a method known in the art.

The brain cells according to the present disclosure may be cultured in culture media that supports the survival or multiplication of the desired type of cells. The culture media may be supplemented with an additive developed for the continuous culture of brain cells. Examples of additives include N2 medium and B27 additive, which are commercially available from Gibco, bovine serum, and the like. The brain cells may be maintained in culture with media exchange. In such a case, the brain cells may be subcultured, as the brain cells continuously multiply and aggregate to form neurospheres. The subculture may be carried out every approximately 7 to 8 days depending on the situation.

The composition according to the present disclosure inhibits the aggregation and phosphorylation of α-synuclein to protect brain cells, including neurons and glia, from damage, thereby allowing neurons to be replenished (regenerated) or reconstructed (restored).

As used herein, the term “regeneration” refers to the supplementation of a lost part of a formed organ or individual. The term “restoration”, which may be called “reconstitution”, refers to reconstruction of tissue, and again constructing tissues or organs from cells or tissues that are dissociated.

The composition or cell therapeutic agent of the present disclosure may be formulated into an appropriate preparation by incorporating an acceptable carrier depending on the administration mode. The preparations suitable to the administration mode are known, and may include preparations that typically pass through a membrane and facilitate migration.

The composition of the present disclosure may also be used in the form of a usual medicinal preparation. A parenteral preparation may be prepared in the form of a sterile aqueous solution, a non-aqueous solvent, a suspending agent, an emulsion, or a freeze-drying agent. For oral administration, the composition may be prepared in the form of a tablet, a troche, a capsule, an elixir, a suspension, a syrup, or a wafer, and for injections, the composition may be prepared into a single-dose ampoule or multi-dose container. The composition for treatment of the present disclosure may be administered together with a pharmaceutically acceptable carrier. For example, for oral administration, a binder, a lubricant, a disintegrator, an excipient, a solubilizer, a dispersant, a stabilizer, a suspending agent, a colorant, a flavor, or the like may be used. For injections, a buffer, a preservative, an analgesic, a solubilizer, an isotonic agent, a stabilizer, or the like may be used. For topical administration, a substrate, an excipient, a lubricant, a preservative, or the like may be used.

In accordance with still another aspect of the present disclosure, there is provided a composition for the prevention or treatment of a disease caused by α-synuclein protein aggregation, the composition containing any one selected from the group consisting of:
a gene carrier containing Nurr1 and Foxa2 genes; and brain cells transduced with Nurr1 and Foxa2 genes.

In accordance with still another aspect of the present disclosure, there is provided a composition for the prevention or treatment of a disease caused by α-synuclein protein aggregation, the composition containing any one selected from the group consisting of:
a vector loading Nurr1 and Foxa2 genes; and brain cells transduced with Nurr1 and Foxa2 genes.

In accordance with still another aspect of the present disclosure, there is provided a method for treating or palliating a disease caused by α-synuclein protein aggregation, the method including:

administering a composition to a subject, the composition containing any one selected from the group consisting of: a gene carrier containing Nurr1 and Foxa2 genes; and brain cells transduced with Nurr1 and Foxa2 genes.

In accordance with still another aspect of the present disclosure, there is provided a method for treating or palliating a disease caused by α-synuclein protein aggregation, the method including:
administering a composition to a subject, the composition containing any one selected from the group consisting of: a viral vector loading Nurr1 and Foxa2 genes; and brain cells transduced with Nurr1 and Foxa2 genes.

As used herein, the “subject” may refer to a vertebrate as a subject of treatment, observation, or experiments, for example, a cow, a pig, a horse, a goat, a dog, a cat, a rat, a mouse, a rabbit, a guinea pig, a human, or the like.

As used herein, the term “treatment” refers to an approach for obtaining beneficial or preferable clinical results. For the purpose of the present disclosure, the beneficial or preferable clinical results encompass, without limitation, the palliation of a symptom, a decrease in the extent of a disease, the stabilization (that is, no worsening) of a disease condition, a delay of disease progression or a decrease in disease progression rate, (partial or overall) improvement, temporary palliation or a relief of a disease condition, the probability of being either detectable or undetectable, and the like. In addition, the term “treatment” may refer to an increase in survival rate compared with an expected survival rate when a subject receives no treatment. The “treatment” indicates all types of methods, such as therapeutic treatment and prophylactic or preventive measures. The treatments include both treatments required for disorders to be prevented and treatments for already developed disorders. The term “palliating” of a disorder refers to reducing an extent of disease condition and/or an undesirable clinical symptom and/or delaying or lengthening a time course of disease progression, compared with the untreated disorders.

In an embodiment of the present disclosure, the disease due to α-synuclein may be selected from the group consisting of Parkinson's disease and dementia with Lewy bodies, but is not limited thereto.

In addition, a method for treating a disease caused by α-synuclein by using the composition for treatment of the present disclosure may include administering to a subject or patient through a typical route into which a predetermined material is introduced, in an appropriate manner.

Examples of the administration method include intracranial administration, intrames-encephalic administration, intraventricular administration, spinal cavity administration, intraperitoneal administration, intravenous administration, intramuscular administration, subcutaneous administration, intradermal administration, oral administration, topical administration, intranasal administration, intrapulmonary administration, and rectal administration, but are not limited thereto.

In addition, the composition according to the present disclosure may also be administered by any device that can deliver an active substance to a target cell. The administration modes and preparations may include a midbrain injection using the stereotactic system, a substantia nigra injection, a cerebral ventricle injection, a cerebrospinal fluid injection, an intravenous injection, a subcutaneous injection, an intradermal injection, an intramuscular injection, or a drop injection. The injections may be prepared by using an aqueous solvent, such as physiological saline solution or Ringer's solution, and a non-aqueous solvent, such as vegetable oil, a higher fatty acid ester (e.g., ethyl oleate, etc.), an alcohol (e.g., ethanol, benzyl alcohol, propylene glycol, or glycerin). The injections may contain a pharmaceutical carrier, such as a stabilizer for deterioration prevention (e.g., ascorbic acid, sodium hydrogen sulfite, sodium pyrosulfite, BHA, tocopherol, EDTA, etc.), an emulsifier, a buffer for pH adjustment, a preservative for inhibiting microbial growth (e.g., phenylmercuric nitrate, thimerosal, benzalkonium chloride, phenol, cresol, benzyl alcohol, etc.), and the like.

The composition according to the present disclosure may be administered at a pharmaceutically effective amount. The pharmaceutically effective amount may be easily determined by a person skilled in the art according to factors well known in the medical field, including the type of disease, age, body weight, health, and sex of a subject (patient), drug sensitivity of a subject (patient), route of administration, method of administration, number of times of administration, duration of treatment, or drug(s) to be mixed or simultaneously used.

The brain cells transduced with Foxa2 and/or Nurr1 genes according to the present disclosure may be directly transplanted in the form of a composition into a lesion site according to the therapeutically effective amount.

As used herein, the term “therapeutically effective amount” refers to an amount sufficient to stop or relieve a physiological effect of a subject or patient, caused by the aggregation or phosphorylation of α-synuclein. The therapeutically effective amount of the cells used may depend on the needs by a subject (patient), age, physiological condition, and health of a subject (patient), a predetermined therapeutic effect, the size and area of the tissue to be targeted for treatment, the severity of a lesion, and a selected route of delivery. In addition, a low dose of cells may be administered to one or more sites in a predetermine target tissue in the form of small multiple grafts. The cells of the present disclosure may be completely isolated before transplantation, for example, to form a suspension of single cells, or may be almost completely isolated before transplantation, for example, to form small cell aggregates. The cells may be administered by transplanting or migrating such a suspension or small cell aggregates to a predetermined tissue site and reconstructing or regenerating a functionally deficient region.

A suitable range of cells to be administered to achieve therapeutic effectiveness may be properly determined for a subject or a patient, within the ordinary skill of a person skilled in the art. For example, the dose of the cells that may be contained in the composition according to the present disclosure may be approximately 10 to 1,000,000,000, but is not limited thereto.

The suitable dose of the composition of the present disclosure may be determined by factors, such as the method of formulation, the manner of administration, age, body weight, or gender of a subject (patient), the severity of a disease symptom, food, the time of administration, the route of administration, excretion rate, and response sensitivity. An ordinarily skilled physician can easily determine and prescribe a dose effective for the desired treatment. The pharmaceutical composition of the present disclosure may contain a viral vector or a viral gene of 1×10¹−1×10¹³ virus genome(vg)/μl, 1×10²−1×10¹³ vg/μl, 1×10³−1×10¹³ vg/μl, 1×10⁴−1×10¹³ vg/μl, 1×10⁵−1×10¹³ vg/μl, 1×10⁶−1×10¹³ vg/μl, 1×10⁷−1×10¹³ vg/μl, 1×10⁸−1×10¹³ vg/μl, 1×10⁹−1×10¹³ vg/μl, 1×10¹⁰−1×10¹³ vg/μl, 1×10¹¹−1×10¹³ vg/μl, 1×10¹²−1×10¹³ vg/μl, 1×10¹−1×10¹² vg/μl, 1×10¹−1×10¹¹ vg/μl, 1×10¹−1×10¹⁰ vg/μl, 1×10¹−1×10⁹ vg/μl, 1×10¹−1×10⁸ vg/μl, 1×10¹−1×10⁷ vg/μl, 1×10¹−1×10⁶ vg/μl, 1×10¹−1×10⁵ vg/μl, 1×10¹−1×10⁴ vg/μl, 1×10¹−1×10³ vg/μl, 1×10¹−1×10² vg/μl, 1×10²−1×10¹² vg/μl, 1×10³−1×10¹¹ vg/μl, 1×10⁴−1×10¹⁰ vg/μl, 1×10⁵−1×10° vg/μl, 1×10⁶−1×10⁸ vg/μl, 1×10²−1×10³ vg/μl, 1×10³−1×10⁴ vg/μl, 1×10⁴−1×10⁵ vg/μl, 1×10⁵−1×10⁶ vg/μl, 1×10⁶−1×10⁷ vg/μl, 1×10⁷−1×10⁸ vg/μl, 1×10⁸−1×10º vg/μl, 1×10⁹−1×10¹⁰ vg/μl, 1×10¹⁰−1×10¹¹ vg/μl, 1×10¹¹−1×10¹² vg/μl. Typically, 1×10⁶ to 2×10¹⁶ vg/dose may be injected into a patient once to five times. For the maintenance of effects, an injection may be again performed by a similar method after several months or years.

### Advantageous Effects of Invention

The present disclosure relates to a composition for inhibiting α-synuclein aggregation and a method for inhibiting α-synuclein aggregation and, more specifically, to techniques for inhibiting α-synuclein aggregation and phosphorylation by introducing Nurr1 and Foxa2 genes to induce the expressions thereo. The composition, according to the present disclosure, has a significant effect on inhibiting α-synuclein aggregation and phosphorylation and thus can be used in the treatment and prevention of Parkinson's disease.

### BRIEF DESCRIPTION OF DRAWINGS

FIG. 1 shows Western blotting results after the control (Cont) and dopamine neurons and glia transduced with Nurr1 and Foxa2 genes (NF) were treated with α-synuclein preformed fibril (PFF) according to an example.

FIG. 2 is a graph comparing the degrees of α-synuclein aggregation in the control and the group transduced with Nurr1 and Foxa2 genes (NF) after Western blotting according to an embodiment.

FIG. 3 is a graph comparing the levels of phosphorylated α-synuclein monomers in the control and the group transduced with Nurr1 and Foxa2 genes (NF) after Western blotting according to an embodiment.

FIG. 4 is a graph comparing the levels of phosphorylated α-synuclein aggregates in the control and the group transduced with Nurr1 and Foxa2 genes (NF) after Western blotting according to an embodiment.

FIG. 5 is an image showing Western blotting results after the control (Cont) and dopamine neurons and glia transduced with Nurr1 gene alone (N), Foxa2 gene alone (F), and both Nurr1 and Foxa2 genes (NF) were treated with α-synuclein preformed fibril (PFF) according to an example.

FIG. 6 is a graph comparing the levels of α-synuclein aggregates and monomers among the control (Con), the Foxa2 alone introduction group (F), the Nurr1 alone introduction group (N), and the Nurr1 and Foxa2 introduction group (NF) after Western blotting according to an example.

FIG. 7 is a graph showing the pole test results for the wild type (WT), the control (Cont) as Parkinson's disease model, and Nurr1 and Foxa2 introduction group (NF) according to an example.

FIG. 8 depicts images showing the pole test results for the wild type (WT), the control (Cont) as Parkinson's disease model, and Nurr1 and Foxa2 introduction group (NF) according to an example.

FIG. 9 is a graph showing the beam test results on Week 8 for the wild type (WT), the control (PD) as Parkinson's disease model, and Nurr1 and Foxa2 introduction group (NF) according to an example.

FIG. 10 is a graph showing the beam test results on Week 12 for the wild type (WT), the control (PD) as Parkinson's disease model, and Nurr1 and Foxa2 introduction group (NF) according to an example.

FIG. 11 depicts images showing the beam test results for the wild type (WT), the control (PD) as Parkinson's disease model, and Nurr1 and Foxa2 introduction group (NF) according to an example.

FIG. 12 is a graph showing the total distance of travel for the wild type (WT), the control (PD) as Parkinson's disease model, and Nurr1 and Foxa2 introduction group (NF) along the open field test (OFT) according to an example.

FIG. 13 is a graph showing the average speed for the wild type (WT), the control (PD) as Parkinson's disease model, and Nurr1 and Foxa2 introduction group (NF) along the open field test according to an example.

FIG. 14 depicts images showing the open field test results for the wild type (WT), the control (PD) as Parkinson's disease model, and Nurr1 and Foxa2 introduction group (NF) according to an example.

FIG. 15 is a graph showing the rotarod test results on Week 8 for the wild type (WT), the control (PD) as Parkinson's disease model, and Nurr1 and Foxa2 introduction group (NF) according to an example.

FIG. 16 is a graph showing the rotarod test results on Week 12 for the wild type (WT), the control (PD) as Parkinson's disease model, and Nurr1 and Foxa2 introduction group (NF) according to an example.

FIG. 17 is an image showing the rotarod test results for the wild type (WT), the control (PD) as Parkinson's disease model, and Nurr1 and Foxa2 introduction group (NF) according to an example.

### BEST MODE FOR CARRYING OUT THE INVENTION

An α-synuclein protein aggregation inhibitor containing any one selected from the group consisting of:
a gene carrier containing Nurr1 and Foxa2 genes; and
brain cells transduced with Nurr1 and Foxa2 genes.

### MODE FOR THE INVENTION

Hereinafter, the present disclosure will be described in more detail by the following examples. However, these examples are used only for illustration, and the scope of the present disclosure is not limited by these examples.

### Example 1: Vector Production

Vectors were constructed for transduction of lentiviruses. Lentiviral vectors expressing Nurr1 or Foxa2 were generated by inserting each cDNA into the multiple cloning site of pCDH (System Biosciences, Mountain View, CA) under the control of a CMV promoter. pGIPZ-shNurr1 and pGIPZ-shFoxa2 lentiviral vectors were purchased from Open Biosystems (Rockford, IL). The empty backbone vectors pCDH or pGIPZ were used as negative controls. Titers of the lentiviruses were determined using a QuickTiter™ HIV Lentivirus quantification kit (Cell Biolabs, San Diego, CA), and 2 ml/6 cm dishes or 200 μl/well (24-well plates) with 10⁶ transducing unit (TU)/ml (60-70 ng/ml) were used for each transduction reaction.

For inducing in vivo expression by stereotaxic injection, AAVs expressing Nurr1 or Foxa2 under the control of the CMV promoter were generated by subcloning the respective cDNAs into pAAV-MCS vector (Addgene, Cambridge, MA). To assess the expression efficiency of the transferred genes, green fluorescence protein (GFP)-expressing AAVs were also generated. Production, separation, and purification of the AAVs (serotype 9 or 2) were performed by the Korea Institute of Science and Technology (Seoul, Korea). AAV titers were determined using a QuickTiter™ AAV quantification kit (Cell Biolabs). Co-expression studies were carried out by infecting cells with mixtures of individual viral preparations (1:1, v:v).

### Example 2: Cell Culture

### 2-1. Ventral Midbrain Neural Progenitor Cell (VM NPC) Cultures

Ventral midbrain neural progenitor cells with dopaminergic potential were seeded at 4×10⁵/well in 6-well plates coated with poly-L-ornithine-fibronectin (PLO-FN). After 24 hours, lentiviruses expressing Nurr1 and Foxa2 under the control of the synapsin promoter were used to transduce ventral midbrain neural progenitor cells with 10⁶ transducing unit (TU)/ml (60-70 ng/ml) (1:1 (v:v)). The lentiviruses expressing GFP as the control were used with the equivalent amounts. Thereafter, the cells were cultured for 3 days to differentiate into neurons.

### 2-2. Ventral Midbrain Glia Cultures

For ventral midbrain glia cultures, mouse ventral midbrain glia were seeded at 3×10⁶ in 100 mm×20 mm culture dishes coated with poly-L-ornithine-fibronectin, and after 24 hours, lentiviruses individually expressing Nurr1 and Foxa2 under the control of the CMV promoter were used to transduce mouse ventral midbrain glia with 10⁶ transducing unit (TU)/ml (60-70 ng/ml) as mixtures (1:1 (v:v)). Control viruses as the control were used with the equivalent amounts. Thereafter, cultures were carried out for 5 days.

### 2-3. Co-Culture

The VM glia cultured in Example 2-2 were seeded in the dopaminergic VM neurons cultured in Example 2-1 at a ratio of 2:1 (VM neurons:VM glia=2:1). Next day, α-synuclein preformed fibrils (PFF) were used to a final concentration of 2 μg/ml, and after 7 days, the level of the α-synuclein protein was investigated by western blotting.

### Example 3: Western Blotting

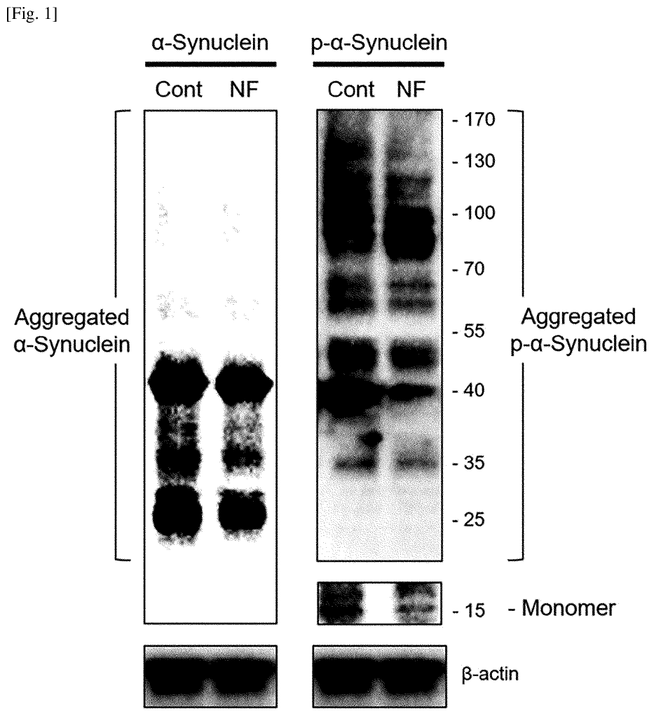

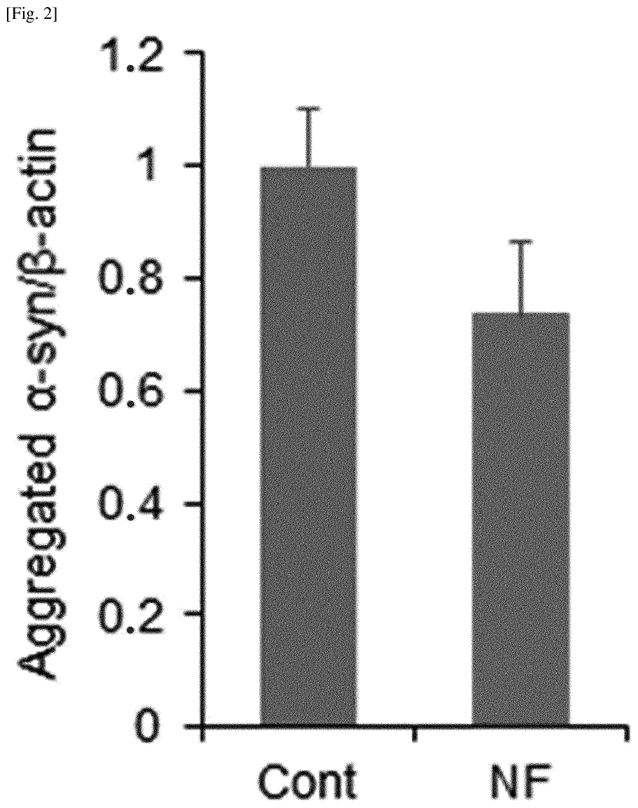

Proteins were extracted by adding protease inhibitor (Roche) and phosphatase inhibitor cocktails (Sigma) in 1% Triton X-100/PBS solution to the plated cells. After centrifugation, the pellet was released in 1% SDS sample buffer, and 15 μg of proteins were loaded onto SDS-PAGE gel (4-16% gradient gel). After transferring to membranes, blocking with 5% BSA/TBST was performed, and primary antibodies were incubated at 4° C. overnight and secondary antibody was incubated at room temperature for 1 hour. The primary antibodies were as follows: α-syn (BD biosciences, 610787); pS129-α-syn (Bio Legend, 825701). The Western blot test results are shown in FIG. 1. After Western blotting, the Western blot results were quantitatively measured using the ImageJ program and shown in FIGS. 2 to 4 and Tables 1 to 3.

TABLE 1

| | Cont | NF |
|---|---|---|
| Aggregated α-syn/β-actin | 1 | 0.73 |

TABLE 2

| | Cont | NF |
|---|---|---|
| Monomeric Pα-syn/β-actin | 1 | 0.34 |

TABLE 3

| | Cont | NF |
|---|---|---|
| Aggregated Pα-syn/β-actin | 1 | 0.62 |

### Example 4: Inhibition of Nurr1 and Foxa2 on α-Synuclein Aggregation and Phosphorylation

As can be seen from the Western blotting results in FIG. 1, the α-synuclein protein aggregation as compared with the control was reduced in the neurons and glia transduced with Nurr1 and Foxa2 genes and treated with α-synuclein PFF compared with the neurons and glia treated without Nurr1 and Foxa2. In addition, the levels of phosphorylated monomers and aggregates of α-synuclein protein were considerably decreased compared with the control.

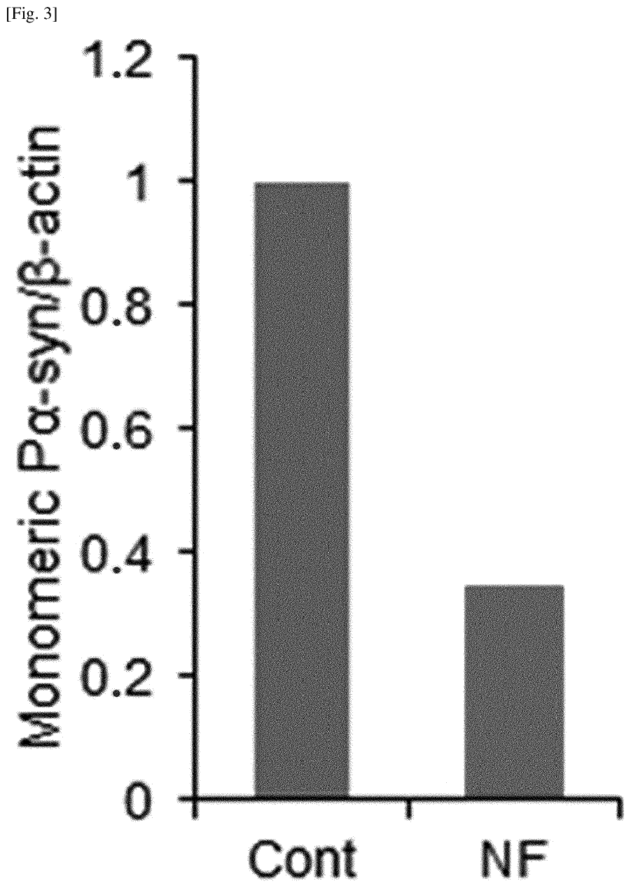

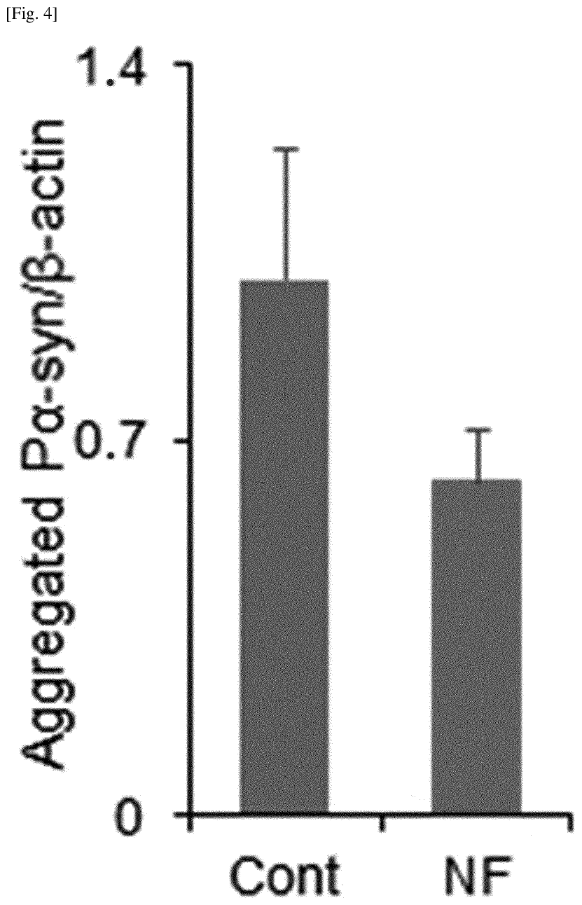

Specifically, as can be confirmed in FIG. 2 and Table 1, when Nurr1 and Foxa2 genes were transduced, the amount and aggregation of aggregated α-synuclein protein were reduced compared with the control. As can be confirmed in FIGS. 3 and 4 and Tables 2 and 3, when Nurr1 and Foxa2 genes were transduced, the level of phosphorylated α-synuclein aggregates was reduced to about 60% of that in the control, and the level of monomers thereof was reduced to about 30%.

### Example 5: Comparison of α-Synuclein Aggregate Formation Inhibitory Effect Between Nurr1 Alone Administration and Nurr1 and Foxa2 Combinative Administration

Neurons and glia transduced with Nurr1 alone, Foxa2 alone, and both Nurr1 and Foxa2 were plated, and proteins were extracted with protease inhibitor (Roche) and phosphatase inhibitor cocktails (Sigma) in 1% Triton X-100/PBS solution.

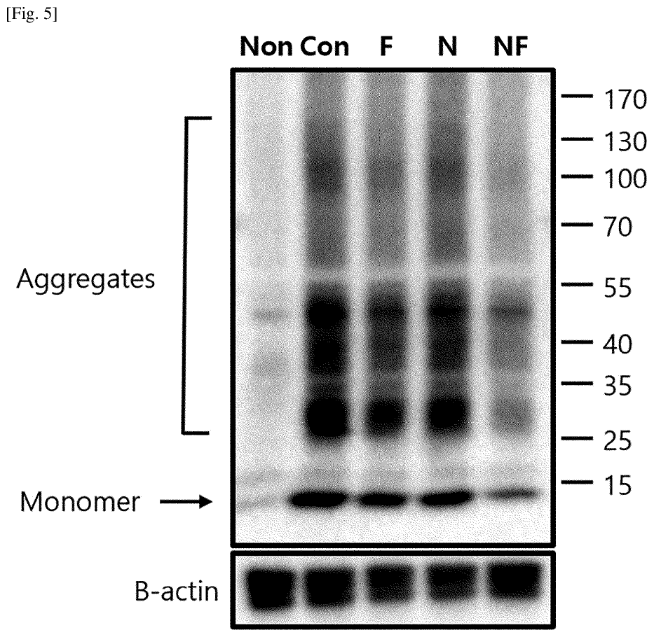

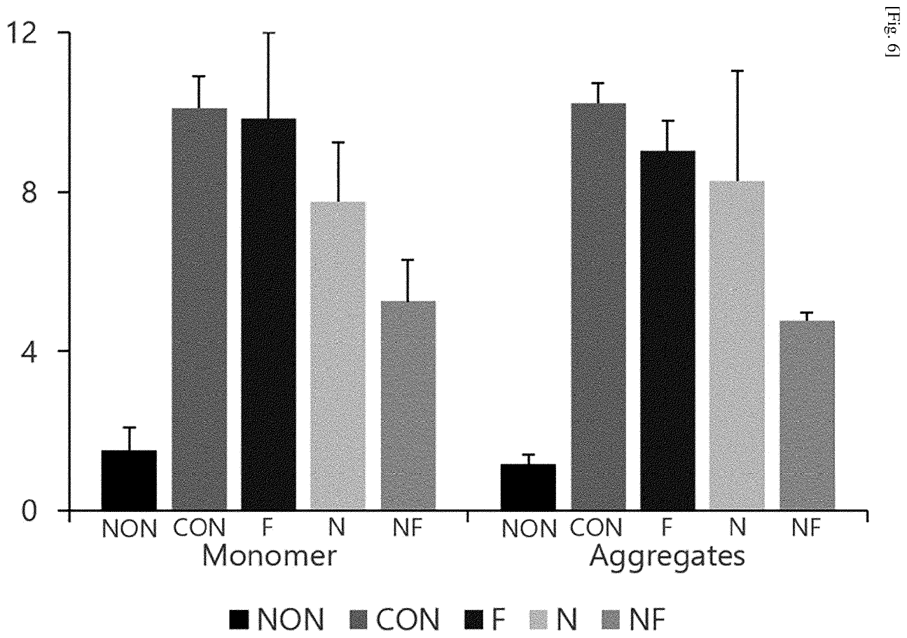

Cells into which Nurr1 or Foxa2 were not transduced were used as the control. After centrifugation, the pellet was released in 1% SDS sample buffer, and 15 μg of proteins were loaded onto SDS-PAGE gel (4-16% gradient gel). After transferring to membranes, blocking with 5% BSA/TBST was performed, and primary antibodies were incubated at 4° C. overnight and secondary antibody was incubated at room temperature for 1 hour. The primary antibodies were as follows: α-syn (BD biosciences, 610787); pS129-α-syn (Bio Legend, 825701). The Western blot test results are shown in FIG. 5. After Western blotting, the Western blot results were quantitatively measured using the ImageJ program and are shown in FIG. 6 and Table 4.

TABLE 4

| | Monomer | | | | | Aggregates | | | | |
|---|---|---|---|---|---|---|---|---|---|---|
| | Non | Con | F | N | NF | Non | Con | F | N | NF |
| Aggregated α- | 1.52 | 10.10 | 9.85 | 7.77 | 5.25 | 1.17 | 10.24 | 9.02 | 8.28 | 4.76 |
| syn/β-actin | | | | | | | | | | |

As can be confirmed from the test results in FIG. 6 and Table 4, the α-synuclein aggregation was significantly reduced in the group transduced with both Nurr1 and Foxa2 genes (N+F) compared with the groups transduced with Nurr1 or Foxa2 alone (N or F). Specifically, the group transduced with both Nurr1 and Foxa2 genes showed a reduction of 48% or more in α-synuclein aggregation compared with the control. Critically, this group showed a reduction of 32% or more in the α-synuclein aggregation compared with the group transduced with Nurr1 alone. From this data, it was determined that the transduction of both Nurr1 and Foxa2 could significantly reduce the α-synuclein aggregation compared with the introduction of Nurr1 alone Furthermore, the introduction of both Nurr1 and Foxa2 genes would be very effective in the treatment of Parkinson's disease.

### Example 6: Behavioral Improvement by Nurr1 and Foxa2 Introduction in Parkinson's Disease Model

### 6-1. Fabrication of Parkinson's Disease (PD) Model

ICR mouse AAV2-CMV-α-syn-HA and α-syn PFF 5 μg were mixed and injected into the substantia nigra (SN) of the mice. Specifically, the mice were fixed by a stereotaxic instrument, and then the skin of the head part was incised along the midline by about 1 cm to confirm the bregma. The skull was drilled using an electric drill at −3.3 mm anterior and 1.2 mm lateral positions with respect to the bregma, and 2 μL of AAV2-CMV-α-syn-HA (1.3×10¹³ gc/μL) and 2 μL of α-syn PFF (5 mg/mL) were loaded in a stereotaxic injector, entered from the skull to a depth of 4.6 mm, and administered with 2 microliters to both sides of the substantia nigra (the dose for each vector for each administration site is 1.3×10¹³ gc/site) at a rate of 0.5 μl/min. After 20 minutes of administration to both sides of the substantia nigra, the injector was pulled back at 1.5 mm per 10 minutes to thereby minimize the leaking of the administered mixture. The skin of the mouse skull was sutured using a medical skin stapler and disinfected with povidone, and the mice were recovered and then placed in a cage.

After four weeks of administration, Nurr1 and Foxa2 were transduced through viral vector(s) expressing Nurr1 and Foxa2. Specifically, the PD mouse model was fixed by a stereotaxic instrument, and then the skin of the head part was incised along the midline by about 1 cm to confirm the bregma. The skull was drilled using an electric drill at −3.3 mm anterior and 1.2 mm lateral positions with respect to the bregma, and AAV9-hNurr1 and AAV9-hFoxa2 were loaded at a concentration of 1×10¹⁰gc/μL for each vector in a stereotaxic injector. The stereotaxic injector entered from the skull to a depth of 4.6 mm, and administered with 2 microliters to both sides of the substantia nigra (the dose for each vector for each administration site is 1.0×10¹⁰ gc/site) at a rate of 0.5 μl/min. After 20 minutes of administration to both sides of the substantia nigra, the injector was pulled back at 1.5 mm per 10 minutes to thereby minimize the leaking of the administered mixture. The skin of the skull was sutured using a medical skin stapler and disinfected with povidone, and the mice were recovered, and then placed in a cage.

### 6-2. Pole Test

Nurr1 and Foxa2 were transduced, and after 4, 8, and 12 weeks, the pole test was performed. The test was performed after the mice were acclimatized by training in advance 2 to 3 days before the test. In the test, the time for the mice to go down from the upper end of the pole was measured for the wild type (WT), the control (Cont) as Parkinson's disease model, and the Nurr1 and Foxa2 transduction group (NF). During the test, the negative value was set when the mouse fell or slipped from the pole and set as the same value as the maximum value in the corresponding week for data generation. Herein, six mice were tested for the wild-type group, and eight mice were tested for the control and Nurr1 and Foxa2 transduction groups. Significant effects for measured times were determined through one-way ANOVA.

TABLE 5

| | Prior to NF | 4 weeks | 8 weeks | 12 weeks |
|---|---|---|---|---|
| | introduction | later | later | later |
| Wild type (WT) | 7.84 | 4.77 | 4.62 | 4.92 |
| Control (Cont) | 10.26 | 10.20 | 8.86 | 9.02 |
| Transduction group | 10.42 | 7.23 | 6.35 | 6.72 |
| (NF) | | | | |

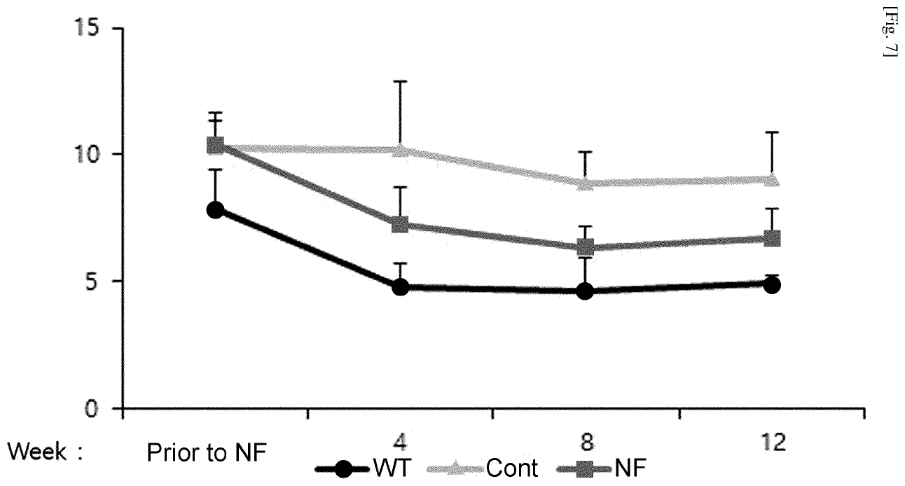

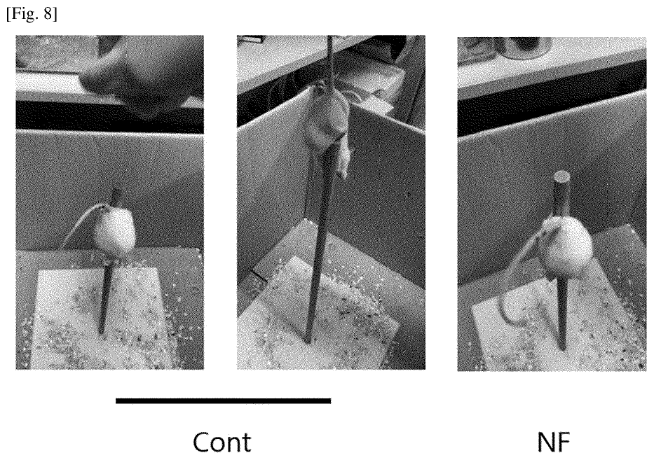

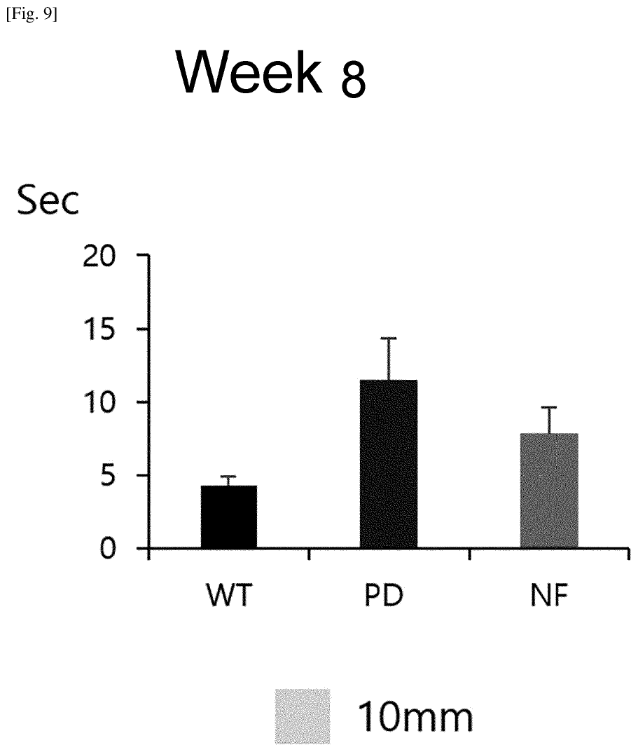

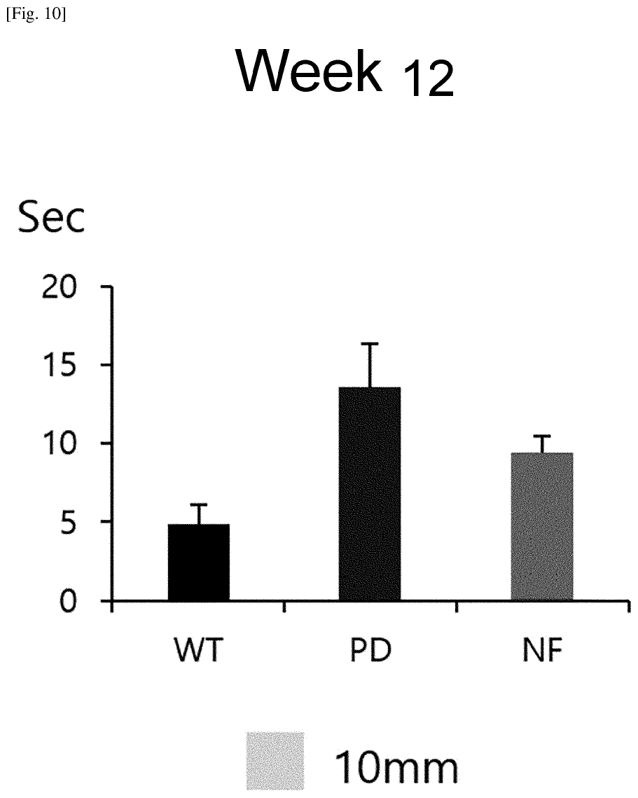

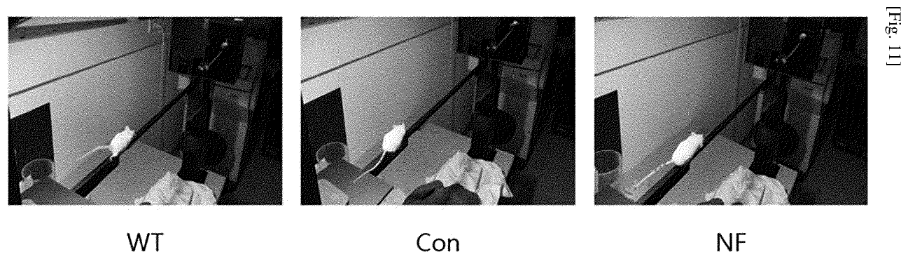

As can be confirmed from the test results in FIGS. 7 and 8 and Table 5, the mice of the control took a longer time to climb down the pole and had a higher frequency of falling and sliding off the pole compared with the mice of the wild-type and the Nurr1 and Foxa2 transduction (NF) group.

### 6-3. Beam Test

Nurr1 and Foxa2 were transduced, and after 8 and 12 weeks, the beam test was performed. The test was performed after the mice were acclimatized by training in advance 2 to 3 days before the test. The beam used in the test was square and 10 mm in thickness. In the test, the time for the mice to traverse from one end to the other end of the beam was measured twice for wild type (WT), the control (PD) as Parkinson's disease model, and the Nurr1 and Foxa2 transduction group (NF). During the test, the negative value was set when the mouse fell or slipped from the beam and set as the same value as the maximum value in the corresponding week for data generation. Herein, six mice were evaluated from the wild-type, control (PD group), and Nurr1 and Foxa2 transduction groups. Significant effects for measured times were determined through one-way ANOVA.

TABLE 6

| | Week 8 | | | Week 12 | | |
|---|---|---|---|---|---|---|
| | WT | PD | NF | WT | PD | NF |
| Time required (sec) | 4.26 | 11.46 | 7.85 | 4.89 | 13.57 | 9.42 |

As can be confirmed from the test results in FIGS. 7 and 8 and Table 6, the mice of the control took a longer time to traverse the beam compared with the mice of the wild-type and the Nurr1 and Foxa2 transduction (NF) group.

### 6-4. Open Field Test (OFT)

Nurr1 and Foxa2 were transduced, and after 8 weeks, the open field test was performed. The mice of the wild type (WT), the control (PD) Parkinson's disease model, and the Foxa2 transduction group (NF) were placed in an open field, and then the travel path, speed, distance, or the like of the mice of each group were measured for 5 minutes. Six mice were evaluated for all of the wild-type, control (PD group), and Nurr1 and Foxa2 transduction group. Significant effects for measured times were determined through one-way ANOVA.

TABLE 7

| | Week 8 | | |
|---|---|---|---|
| | WT | PD | NF |
| Total distance (m) | 12.84 | 5.91 | 12.95 |

TABLE 8

| | Week 12 | | |
|---|---|---|---|
| | WT | PD | NF |
| Average speed (m/sec) | 0.042 | 0.019 | 0.043 |

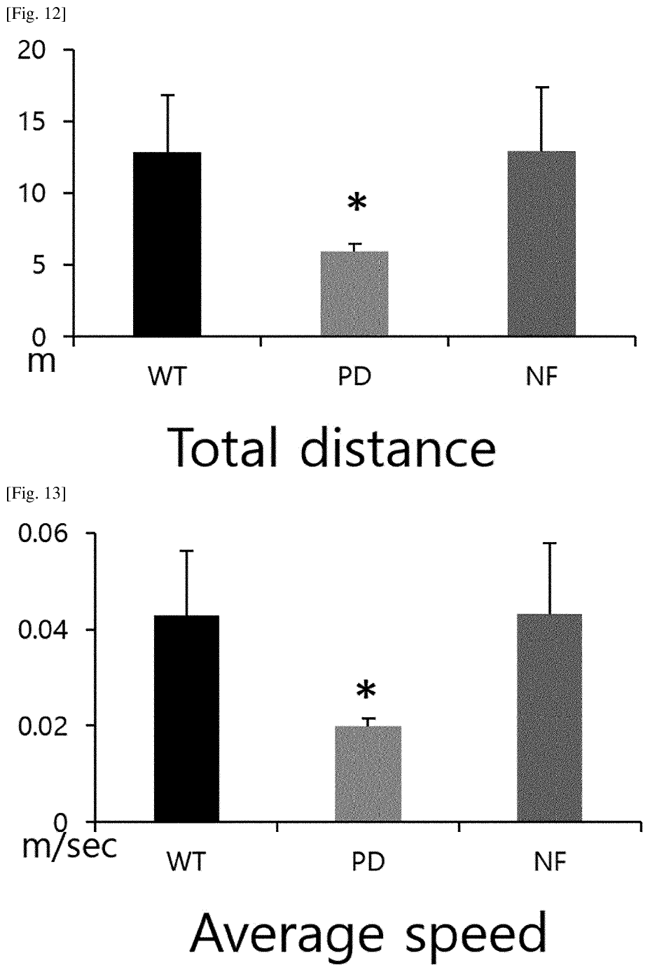

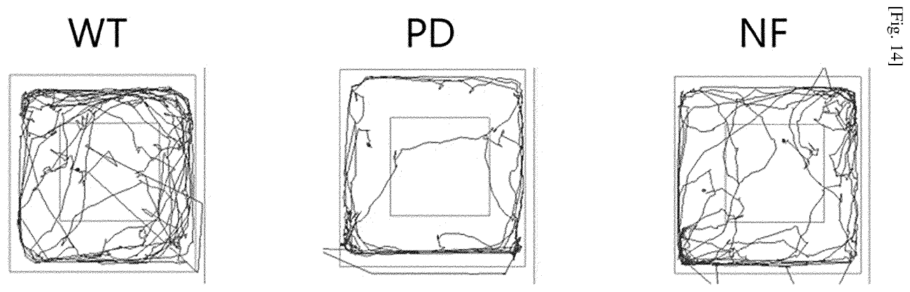

As can be confirmed from the test results in FIGS. 12 to 14 and Tables 7 and 8, the mice of the Nurr1 and Foxa2 transduction group (NF) group showed behavioral improvement to the extent that is almost no difference in total travel distance and average speed compared with the wild-type mice. The mice of the Nurr1 and Foxa2 transduction group (NF) group showed a significantly increased total distance and average speed of travel compared with the control (PD).

### 65. Rotarod Test

Nurr1 and Foxa2 were transduced, and after 8 and 12 weeks, the rotarod test was performed. The test was performed after the mice were acclimatized by training in advance 2 to 3 days before the test. In the test, the time for the mice to fall off from the rod with a slowly increasing rate from 4 rpm to 40 rpm was measured twice for the wild type (WT), the control (PD) Parkinson's disease model, and the Nurr1 and Foxa2 transduction group (NF). Six mice were evaluated for all of the wild-type, control (PD group), and Nurr1 and Foxa2 transduction groups. Significant effects for measured times were determined through one-way ANOVA.

TABLE 9

| | Week 8 | | |
|---|---|---|---|
| | WT | PD | NF |
| Time required (sec) | 195.45 | 71.40 | 136.40 |

TABLE 10

| | Week 12 | | |
|---|---|---|---|
| | WT | PD | NF |
| Time required (sec) | 211.3 | 89.58 | 176.58 |

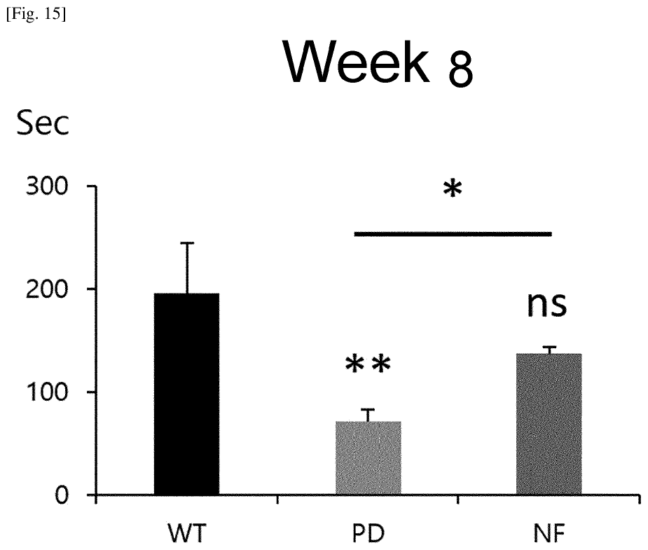

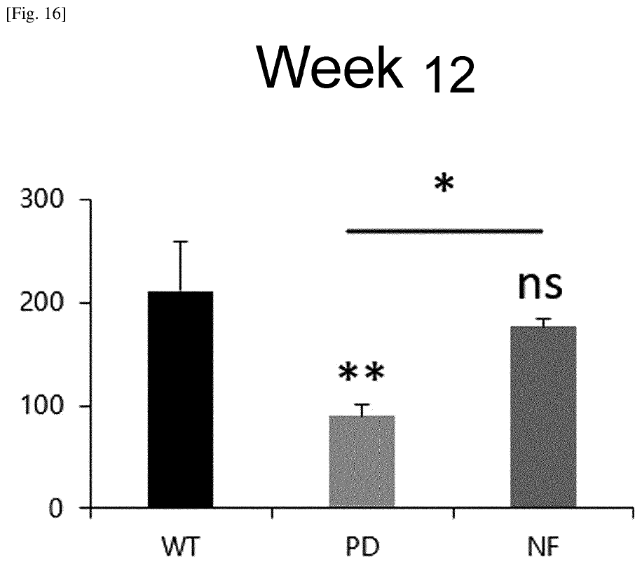

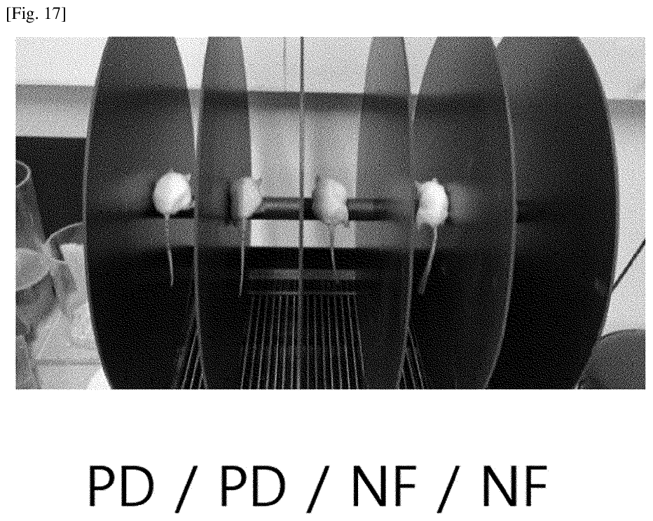

As can be confirmed from the test results in FIGS. 15 to 17 and Tables 9 and 10, the mice of the Nurr1 and Foxa2 transduction group (NF) group took a significantly longer time to fall off the rod compared with the control on Week 8 and Week 12.

6−6. Conclusion

As such, it was identified that the mice of the Nurr1 and Foxa2 transduction group (NF) significantly improved motor ability and behavior in all of the pole test, beam test, open field test, and rotarod test, compared with the control group corresponding to the Parkinson's disease model. It was identified through the above results that the transduction of Nurr1 and Foxa2 genes can restore stiffness, bradykinesia, and postural instability, which are characteristics of Parkinson's disease. Accordingly, the transduction of Nurr1 and Foxa2 genes is expected to become a therapeutic option in the treatment of Parkinson's disease.

### INDUSTRIAL APPLICABILITY

The present disclosure relates to a composition for inhibiting α-synuclein aggregation and a method for inhibiting α-synuclein aggregation and, more specifically, to techniques for inhibiting α-synuclein aggregation and phosphorylation by introducing Nurr1 and Foxa2 genes to induce the expressions thereof.

### INDUSTRIAL APPLICABILITY

The present disclosure relates to a composition for inhibiting α-synuclein aggregation and a method for inhibiting α-synuclein aggregation and, more specifically, to techniques for inhibiting α-synuclein aggregation and phosphorylation by introducing Nurr1 and Foxa2 genes to induce the expressions thereof.

1. An alpha-synuclein (α-synuclein) protein aggregation inhibitor comprising any one selected from the group consisting of:
a gene carrier containing Nurr1 and Foxa2 genes; and
brain cells introduced with Nurr1 and Foxa2 genes.

2. The α-synuclein protein aggregation inhibitor of claim 1, wherein the gene carrier is a viral vector.

3. The α-synuclein protein aggregation inhibitor of claim 2, wherein the viral vector is one selected from the group consisting of an adeno-associated viral vector, a retroviral vector, and an adenoviral vector.

4. The α-synuclein protein aggregation inhibitor of claim 1, wherein the brain cells are neurons, and glia including astrocytes or microglia.

5. An α-synuclein protein phosphorylation inhibitor comprising any one selected from the group consisting of:
a gene carrier containing Nurr1 and Foxa2 genes; and
brain cells introduced with Nurr1 and Foxa2 genes.

6. The α-synuclein protein phosphorylation inhibitor of claim 5,
wherein the gene carrier is a viral vector.

7. The α-synuclein protein phosphorylation inhibitor of claim 6,
wherein the viral vector is one selected from the group consisting of an adeno-associated viral vector, a retroviral vector, and an adenoviral vector.

8. The α-synuclein protein phosphorylation inhibitor of claim 5,
wherein the brain cells are neurons, and glia including astrocytes or microglia.

9. A composition for the prevention or treatment of a disease caused by α-synuclein protein aggregation or phosphorylation, the composition comprising any one selected from the group consisting of:
a gene carrier containing Nurr1 and Foxa2 genes; and
brain cells introduced with Nurr1 and Foxa2 genes.

10. The composition of claim 9, wherein the gene carrier is a viral vector.

11. The composition of claim 10, wherein the viral vector is one selected from the group consisting of an adeno-associated viral vector, a retroviral vector, and an adenoviral vector.

12. The composition of claim 9, wherein the brain cells are neurons, and glia including astrocytes or microglia.

13. The composition of claim 9, wherein the disease caused by α-synuclein protein aggregation is selected from the group consisting of Parkinson's disease and dementia with Lewy bodies.

14. A method for treating a disease caused by α-synuclein protein aggregation or phosphorylation, the method including:
administering to a subject in need thereof the composition of claim 9.
